(19) United States
(12) Patent Application Publication
(10) Pub. No.: US 2005/0214265 A1
(43) Pub. Date: Sep. 29, 2005

(54) Method for improving bone marrow engraftment

(76) Inventor: Suzanne T. IIdstad, Prospect, KY (US)

Correspondence Address: HOGAN & HARTSON LLP, ONE TABOR CENTER, SUITE 1500, 1200 SEVENTEENTH ST, DENVER, CO, 80202, US

(21) Appl. No.: 10/808,205

(22) Filed: Mar. 24, 2004

(51) Int. Cl. A61K 48/00; A61K 31/66; A61K 39/395

(52) U.S. Cl. 424/93.21; 424/140.1; 514/109; 514/44

(57) ABSTRACT

The present invention demonstrates that a delay in marrow infusion is associated with a decrease in the amount of conditioning required and improved engraftment. Surprisingly, it has been discovered that for a given dose of TBI, a delay in transplantation of the marrow for up to 8 days, and preferably 5 days, following conditioning was associated with a higher proportion of animals engrafting. For a given dose of conditioning, the highest proportion of animals engrafted when the marrow infusion was delayed by 4 days. In recipients conditioned with 700 cGy of TBI, a delay of marrow infusion for up to 4 days did not impair the establishment of chimerism. In recipients treated with 600 cGy TBI, engraftment was significantly improved if the marrow was infused 1-4 days following conditioning compared to the conventional delay of approximately 6 hours.

This research was supported in part by the National Institutes of Health, grant DK43901-08. The government has certain rights in the invention.

### BACKGROUND OF THE INVENTION

1. Field of the Invention

The present invention relates to methods for improving the rate of engraftment of solid-organ grafts. More specifically, the present invention relates to non-lethal methods of conditioning a recipient for bone marrow transplantation followed by a delayed transplantation of bone marrow, wherein the transplantation takes place at some period of time between 0 and 8 days following the conditioning of the recipient.

2. Description of the State of Art

The transfer of living cells, tissues, or organs from a donor to a recipient, with the intention of maintaining the functional integrity of the transplanted material in the recipient defines transplantation. Transplants are categorized by site and genetic relationship between the donor and recipient. An autograft is the transfer of one's own tissue from one location to another; a syngeneic graft (isograft) is a graft between identical twins; an allogeneic graft (homograft) is a graft between genetically dissimilar members of the same species; and a xenogeneic graft (heterograft) is a transplant between members of different species.

A major goal in solid organ transplantation is the permanent engraftment of the donor organ without a graft rejection immune response generated by the recipient, while preserving the immunocompetence of the recipient against other foreign antigens. Typically, in order to prevent host rejection responses, nonspecific immunosuppressive agents such as cyclosporine, methotrexate, steroids and FK506 are used. These agents must be administered on a daily basis and if stopped, graft rejection usually results. However, a major problem in using nonspecific immunosuppressive agents is that they function by suppressing all aspects of the immune response, thereby greatly increasing a recipient's susceptibility to opportunistic infections, rate of malignancy, and end-organ toxicity. The side effects associated with the use of these drugs include opportunistic infection, an increased rate of malignancy, and end-organ toxicity (Dunn, D. L., Crit. Care. Clin., 6:955 (1990)). Although immunosuppression prevents acute rejection, chronic rejection remains the primary cause of late graft loss (Nagano, H., et al., Am. J. Med. Sci, 313:305-309 (1997)).

For every organ, there is a fixed rate of graft loss per annum. The five-year graft survival for kidney transplants is 74% (Terasaki, P. I., et al., UCLA Tissue Typing Laboratory (1992)). Only 69% of pancreatic grafts, 68% of cardiac transplants and 43% of pulmonary transplants function 5 years after transplantation (Opelz, G., Transplant Proc, 31:31S-33S (1999)).

The only known clinical condition in which complete systemic donor-specific transplantation tolerance occurs is when chimerism is created through bone marrow transplantation. (See, Qin, et al., J. Exp. Med., 169:779 (1989); Sykes, et al., Immunol. Today, 9:23-27 (1988); and Sharabi, et al., J. Exp. Med., 169:493-502 (1989)). This has been achieved in neonatal and adult animal models as well as in humans by total lymphoid or body irradiation of a recipient followed by bone marrow transplantation with donor cells. The success rate of allogeneic bone marrow transplantation is, in large part, dependent on the ability to closely match the major histocompatability complex (MHC) of the donor cells with that of the recipient cells to minimize the antigenic differences between the donor and the recipient, thereby reducing the frequency of host-versus-graft responses and graft-versus-host disease (GVHD). In fact, MHC matching is essential, only a one or two antigen mismatch is acceptable because GVHD is very severe in cases of greater disparities.

The major histocompatability complex (MHC) is a cluster of closely linked genetic loci encoding three different classes (class I, class II, and class III) of glycoproteins expressed on the surface of both donor and host cells that are the major targets of transplantation rejection immune responses. The MHC is divided into a series of regions or subregions and each region contains multiple loci. An MHC is present in all vertebrates, and the mouse MHC (commonly referred to as H-2 complex) and the human MHC (commonly referred to as the Human Leukocyte Antigen or HLA) are the best characterized.

The role of MHC was first identified for its effects on tumor or skin transplantation and immune responsiveness. Different loci of the MHC encode two general types of antigens which are class I and class II antigens. In the mouse, the MHC consists of 8 genetic loci: Class I is comprised of K and D, class II is comprised of I-A and/or I-E. The class II molecules are each heterodimers, comprised of I-Aα and I-Aβ and/or I-Eα and I-Eβ. The major function of the MHC molecule is immune recognition by the binding of peptides and the interaction with T cells, usually via the αβ T-cell receptor. It was shown that the MHC molecules influence graft rejection mediated by T cells (Curr. Opin. Immunol., 3:715 (1991)), as well as by NK cells (Annu. Rev. Immunol., 10:189 (1992); J. Exp. Med., 168:1469 (1988); Science, 246:666 (1989)). The induction of donor-specific tolerance by HSC chimerism overcomes the requirement for chronic immunosuppression. See, Ildstad, S. T., et al., Nature, 307:168-170 (1984); Sykes, M., et al., Immunology Today, 9:23-27 (1998); and Spitzer, T. R., et al., Transplantation, 68:480-484 (1999)). Moreover, bone marrow chimerism also prevents chronic rejection (Colson, Y., et al., Transplantation, 60:971-980 (1995); and Gammie, J. S., et al., In Press Circulation (1998)). The association between chimerism and tolerance has been demonstrated in numerous animal models including rodents, (Ildstad, S. T., et al., Nature, 307:168-170 (1984); and Billingham, R. E., et al., Nature, 172:606 (1953)) large animals, primates and humans (Knobler, H. Y., et al., Transplantation, 40:223-225 (1985); and Sayegh, M. H., et al., Annals of Internal Medicine, 114:954-955 (1991)).

The cells of all hematopoietic lineages are produced by hematopoietic stem cells (HSC). During this procedure, some HSC retain a long-term multilineage repopulating potential (self-renewal); and some HSC may only retain a short-term multilineage repopulating potential and differentiate to produce progeny. The major purified HSC transplantation-related complications include graft rejection and graft failure. The outcome for engraftment of highly purified HSC in the major histocompatability complex (MHC)-matched recipients is different from that for MHC-disparate allogeneic recipients (El-Badri, N. S., Good, R. A., (1993); and Kaufman, C. L., S. Cell Biochem Suppl., 18:A112 (1994)).

The hematopoietic microenvironment plays a major role in the engraftment of HSC. In addition to being a source of growth factors and cellular interactions for the survival and renewal of stem cells, it may also provide physical space for these cells to reside. A number of cell types collectively referred to as stromal cells are found in the vicinity of the hematopoietic stem cells in the bone marrow microenvironment. These cells include both bone marrow-derived CD45⁺ cells and non-bone marrow-derived CD45⁻ cells, such as adventitial cells, reticular cells, endothelial cells and adipocytes.

Recently, Ildstad, et al., identified another bone marrow-derived cell type known as hematopoietic facilitatory cells, which when co-administered with donor bone marrow cells enhance the ability of the donor cells to stably engraft in allogeneic and xenogeneic recipients. See, U.S. Pat. No. 5,772,994 which is incorporated herein by reference. The facilitatory cells and the stromal cells occupy a substantial amount of space in a recipient's bone marrow microenvironment, which may present a barrier to donor cell engraftment. Hematopoietic stem cells bind to facilitatory cells in vitro and in vivo. Thus, the facilitatory cells may provide physical space or niche on which the stem cells survive and are nurtured. It is therefore believed to be desirable to develop conditioning regimens to specifically target and eliminate these and other stromal cell populations in order to provide the space necessary for the hematopoietic stem cells and the associated facilitatory cells in a donor cell preparation to engraft without the use of lethal irradiation. See, U.S. Pat. Nos. 5,635,156 and 5,876,692 which are also incorporated herein by reference.

Until recently it was believed that ablation of the recipient was a prerequisite to achieve engraftment of MHC-disparate bone marrow. More recently, engraftment of allogeneic marrow stem cells has been achieved using nonmyeloablative conditioning (Colson, Y. L., et al., J Immunol, 157: 2820 (1996); Mayumi, H., et al., Transplant Proc, 20: 139 (1988); Wood, M. L., Transplantation, 46: 449 (1988); Cobbold, S. P., Nature, 323: 164 (1986); and Sharabi, Y., J Exp Med, 169: 493 (1989)). It has historically been hypothesized that repair of the host mieroenvironment begins almost immediately following the conditioning and that the marrow should be infused promptly following conditioning. In the case of vertebral marrow harvested from deceased donors for tolerance induction, the preparation of the marrow can result in a delay in marrow infusion of up to 24 hours. One would predict that repair would occur even more rapidly in nonmyeloablatively conditioned recipients. Now, a number of sublethal conditioning approaches in an attempt to achieve engraftment of allogeneic bone marrow stem cells with less aggressive cytoreduction have been reported in rodent models (Mayumi and Good, J. Exp. Med., 169:213 (1989); Slavin, et al., J. Exp. Med., 147(3):700 (1978); McCarthy, et al., Transplantation, 40(1):12 (1985); Sharabi, et al., J. Exp. Med., 172(1):195 (1990); and Monaco et al., Ann. NY Acad. Sci, 129:190 (1966)).

Conditioning of mice with 700 cGy TBI allows durable engraftment of MHC-plus-minor-antigen disparate allogeneic marrow in 100% of recipients (Colson, Y. L., et al., J Immunol, 155: 4179 (1995)). The TBI dose can be reduced to 500 cGy by adding 200 mg/kg of cyclophosphamide 2 days after bone marrow transplantation (BMT) (Colson, Y. L., et al., J Immunol, 155: 4179 (1995)) and to 200 cGy with the addition of antilymphocyte globulin (ALG) to the conditioning approach (Colson, Y. L., et al., J Immunol, 157: 2820 (1996)). In these previous studies, the marrow was infused 4 to 6 hours following conditioning with TBI. However, reliable and stable donor cell engraftment as evidence of multilineage chimerism was not demonstrated, and long-term tolerance has remained a question in many of these models (Sharabi and Sachs, J. Exp. Med., 169:493 (1989); Cobbold, et al., Immunol. Rev., 129:165 (1992); and Qin, et al., Eur. J. Immunol., 20:2737 (1990)). The requirement for lethal or sublethal irradiation of the host which renders it totally or partially, respectively, immunocompetent however, poses a significant limitation to the potential clinical application of bone marrow transplantation to a variety of disease conditions, including solid organ or cellular transplantation, sickle cell anemia, thalassemia and aplastic anemia.

The risk inherent in tolerance-inducing conditioning approaches must be low when less toxic means of treating rejection are available or in cases of morbid, but relatively benign conditions.

Therefore, there remains a need for transplantation procedures that utilize non-lethal methods of conditioning a recipient for bone marrow transplantation that result in stable chimerism and long term-donor-specific tolerance.

### SUMMARY OF THE INVENTION

The present invention is based on the discovery that a delay in bone marrow transplantation for up to 8 days significantly enhances the establishment of chimerism and tolerance in nonmyeloablatively conditioned recipients and reduces the conditioning required. This is associated with significantly suppressed host alloreactivity and a significant reduction in IL-6 levels in the recipient microenvironment. In addition, the enhanced engraftment correlated inversely with the absolute number of recipient NK and NK/T cells in the host environment.

The present invention further provides methods for treating a variety of diseases and disorders with minimal morbidity.

Additional objects, advantages, and novel features of this invention shall be set forth in part in the description and examples that follow, and in part will become apparent to those skilled in the art upon examination of the following or may be learned by the practice of the invention. The objects and the advantages of the invention may be realized and attained by means of the instrumentalities and in combinations particularly pointed out in the appended claims.

To achieve the foregoing and in accordance with the purposes of the present invention, as embodied, the method of this invention comprises infusing bone marrow into a conditioned recipient between 0 and 8 days following conditioning of the recipient.

### BRIEF DESCRIPTION OF THE DRAWINGS

The accompanying drawings, which are incorporated in and form a part of the specifications, illustrate the preferred embodiments of the present invention, and together with the description serve to explain the principles of the invention.

In the Drawings:

FIG. 1 graphically demonstrates the influence of delay of bone marrow infusion following conditioning on engraftment. B10 (H2^{b}) mice were conditioned with 700 cGy, 600 cGy or 500 cGy of TBI and transplanted with 15×10⁶ B 10.BR (H₂^{k}) BM cells at 0-12 days following conditioning. Engraftment was determined by flow cytometric analysis of peripheral blood lymphocytes 28 days after transplantation. This figure shows the percentage of animals with engraftment after BMT. The number of animals for each group was: 700, 600 and 500 cGy: 0 hr, n=6, 6 and 6; 6 hr, n=6, 15 and 6; day 1, n=6, 10 and 6 day 2, n=7, 10 and 7; day 3, n=6, 10 and 6; day 4, n=6, 10 and 6; day 5, n=11, 10 and 6; day 8, n=11, 10 and 6; and day 12, n=10, 10 and 6, respectively.

FIG. 2 graphically demonstrates the influence of cell dose on engraftment in partially conditioned recipients. B10 (H₂^{b}) mice were conditioned with 500 cGy TBI on day 0 and reconstituted with 30×10⁶ BMC from B10.BR (H2^{k}) donors on day 0 (6 hr post TBI), or day +1, +2, +3, +4, +5, +8 or +12 (n=5 per group). FIG. 2A shows the percentage of chimeric animals at 1 month, 2 months, and 6 months post transplantation. FIG. 2B shows the percentage of donor chimerism for each animal at 1 month, 2 months, and 6 months (diamonds). Mean chimerism for each group is indicated by the bar (—).

FIG. 3 graphically demonstrates the influence of cell dose on engraftment. B10 (H2^{b}) mice were conditioned with 500 cGy TBI on day 0 and reconstituted with 45×10⁶ BMC from B10.BR donors on day 0 (6 hr post TBI), +1, +2, +3, +4, +5, +8 and +12 (n=5 per group). 3A): The percentage of recipients with donor chimerism at 1, 2, and 6 months. 3B): The percentage chimerism for individual animals is shown for each time point (diamonds) and the mean chimerism for each group is indicated by the bar (—).

FIG. 4 provides evidence for multilineage engraftment in allogeneic chimeras. Multilineage PBL typing was performed between 5 to 6 months post BMT in durable chimeras. The x-axis shows staining with fluorescein-conjugated antibody against donor class I antigen (H2K^{k}). On the y axis the staining for the different lineages is shown: T cells (αβ-TCR, CD8, CD4), NK cells (NK1.1), B cells (B220), macrophages (Mac-1), and granulocytes (Gr-1). One representative chimera is presented from a total of 6 that were analyzed.

FIG. 5 graphically demonstrates the survival of donor-specific (B10.BR) and MHC-disparate third-party (BALB/c) pancreatic islet allografts in chimeric mice. Diabetes was induced by intravenous injection of Streptozocin (200 mg/kg) in chimeras and in controls that had initially engrafted but subsequently lost their chimerism. Each animal received approximately 600 mouse islets transplanted beneath the left renal capsule. Animals of group A were followed>160 days after transplantation and euthanized at the time with urine glucose test negative. One animal died at 133 days after islet transplantation with urine glucose test negative.

FIG. 6 graphically demonstrates the cellular subpopulations remaining in the host marrow microenvironment following irradiation were characterized by 4-color staining on day 0, 1, 2, 3, 4 and 5 post 500 cGy TBI. At the time of the nadir, marrow from 2 animals was pooled to provide an informative analysis. Data present mean of recipient hematopoiesis among various lineages in 3 animals or 3 groups of animals at each time point. 6A): The total cell number recovered at indicated times; 6B): number of B cells recovered; 6C): CD8⁺, CD4⁺ or NK1.1⁺ cells; or 6D) NK/T cells (TCR⁺/CD3⁺/NK1.1⁺), NK cells (NK1.1⁺/TCR⁻/CD3⁻) or CD3⁺/CD8⁺/TCR⁺ T cells.

FIG. 7 graphically demonstrates mixed lymphocyte reaction (MLR). Splenocytes were harvested from B10 mice irradiated with 500 cGy TBI at 1 hr, 2 days or 4 days post TBI and co-cultured with irradiated (2,000 cGy) autologous (B 10) and allogeneic (B10.BR) stimulator cells. Values are shown as mean ±SD of triplicate cultures in a 1:1 responder to stimulator ratio from a representative experiment. Experiments were performed at least twice and there were 3 samples at each time point.

FIG. 8 graphically demonstrates cytokine enumeration. B10 mice were conditioned with 500 cGy TBI and serum was collected at 6 hours, 2 days or 4 days post TBI. Unmanipulated controls were also prepared. The cytokine milieu was evaluated using the Luminex¹⁰⁰™ multiple immunoassay. Production of each cytokine is represented in pg/ml. There were 3 samples at each time point and each sample was run in duplicate.

### DETAILED DESCRIPTION OF THE PREFERRED EMBODIMENT

The present invention is founded on the discovery that a delay in bone marrow infusion for up to 8 days significantly enhances the establishment of chimerism and tolerance in nonmyeloablatively conditioned recipients and reduces the conditioning required. This is associated with significantly suppressed host alloreactivity and a significant reduction in IL-6 levels in the recipient microenvironment. In addition, the enhanced engraftment correlated inversely with the absolute number of recipient NK and NK/T cells in the host environment. An understanding of the mechanism of conditioning will allow strategies to enable the widespread clinical application of chimerism to induce tolerance.

Thus, the present invention relates to non-lethal methods of conditioning a recipient, which may be any mammal and preferably human, for bone marrow transplantation followed by a delay of between 0 and 8 days of infusing marrow into the preconditioned recipient. Methods of conditioning a recipient are disclosed in U.S. publication No. 2003-0017152 A1, which is incorporated herein by reference and includes the use of non-lethal doses of irradiation, anti-sense DNA technology, cell type-specific antibodies, cell-type specific cytotoxic drugs or a combination thereof. While all known methodologies of conditioning a recipient are useful in the present invention conditioning methodologies the encompass an approach to make space in a recipient's bone marrow by targeting only critical cell populations in the hematopoietic microenvironment are particularly.

As discussed in further detail below, animals deficient in the production of αβ- and γδ-TCR⁺ T-cells are significantly enhanced in their ability to accept allogeneic bone marrow grafts compared to immunocompetent controls. Mice deficient in production of αβ-TCR⁺ cells alone exhibit similar enhanced engraftment only if cyclophosphamide is administered two days after bone marrow transplant (BMT) after conditioning with 300 cGy total body irradiation (TBI). Mice lacking of production of γδ-TCR⁺ T-cells exhibit enhanced engraftment, although to a lesser extent than αβ-TCR⁺ cells, demonstrating that these cells in the host also play a role in resistance to allogeneic bone marrow engraftment. This finding is supported by the fact that only mice deficient in production of αβ and γδ cells (TCR-β/δ KO) reliably engraft with low TBI dose alone or even no TBI without requiring cyclophosphamide, confirming that both αβ- and γδ-TCR⁺ cells in the host function in a nonredundant and critical fashion in alloresistance to engagement. These data therefore implicate αβ plus γδ T-cells rather than NK cells as the primary effector cells in marrow graft rejection in allotransplantation. Therefore, it is contemplated that one can avoid myelotoxic agents and use novel approaches to control T-cell function in the host.

Methods for targeting αβ-TCR⁺ and γδ-TCR⁺ in the recipient hematopoietic environment are discussed in further detail below and therefore the present invention encompasses and contemplates the use of antibodies, antisense DNA technology and non-lethal doses of irradiation as ways of depleting and preferably eliminating αβ-TCR⁺ and γδ-TCR⁺ cells in the recipient hematopoietic environment.

Various procedures known in the art may be used for the production of polyclonal antibodies to antigens of cells making up the hematopoietic microenvironment of the host, including but is not limited to αβ-TCR⁺, γδ-TCR⁺, and/or CD8⁺ cells. For the production of antibodies, various host animals can be immunized by injection with purified or partially purified hematopoietic cells such as stromal cells including but not limited to rabbits, hamsters, mice, rats, etc. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, Ricin and potentially useful human adjuvants such as BCG (bacille Calmette-Guerin) and Corynebacterium parvum.

A monoclonal antibody to antigens of αβ-TCR⁺, γδ-TCR⁺, and/or CD8⁺ cells may be prepared by using any technique which provides for the production of antibody molecules by continuous cell lines in culture. These include but are not limited to the hybridoma technique originally described by Kohler and Milstein, Nature, 256: 495-497 (1975), and the more recent human B-cell hybridoma technique (Kosbor, et al., Immunology Today, 4:72 (1983); Cote, et al., Proc. Natl Acad. Sci., USA, 80:2026-2030 (1983) and the EBV-hybridoma technique (Cole, et al., Monoclonal Antibodies and Cancer Therapy, Alan R. Liss, Inc., pp. 77-96 (1985)). Techniques developed for the production of “chimeric antibodies” by splicing the genes from a mouse antibody molecule of appropriate antigen specificity together with genes from a human antibody molecule can be used (e.g., Morrison, et al., Proc. Natl. Acad. Sci. USA, 81:6851-6855 (1984); Neuberger, et al., Nature, 312:604-608 (1984); Takeda, et al., Nature, 314:452-454 (1985). Such chimeric antibodies are particularly useful for in vivo administration into human patients to reduce the development of host anti-mouse response. In addition, techniques described for the production of single chain antibodies (U.S. Pat. No. 4,946,778, which is incorporated herein by reference) can also be adapted.

Such antibody conjugates may be administered to a human patient prior to or simultaneously with donor cell engraftment. It is preferred that these conjugates are administered intravenously. Although the effective dosage for each antibody must be titrated individually, most antibodies may be used in the dose range of 0.1 mg/kg-20 mg/kg body weight.

In cases where sub-lethal doses of irradiation are used, total body irradiation (TBI) of a human recipient may be administered up to 7.5 Gy as a single dose or a combined total of 22 Gy administered in fractionated doses. Alternatively, TBI may be administered up to about 5.5 Gy.

The use of antisense strategies presents a theoretically simple tool to identify, with exquisite precision, the molecular mechanisms responsible for various cellular processes. It is based on the fact that each protein synthesized by a cell is encoded by a specific messenger mRNA (mRNA). If translation of a specific RNA is inhibited, the protein product derived from this translation will likewise be reduced. Oligonucleotide sequences, can therefore be designed to be complementary (antisense) to a specific target mRNA sequence, such as the β-chain and/or the δ-chain of TCR, and because of this complementarity, it will bind to the target sequence thereby inhibiting translation of that specific mRNA. An antisense oligonucleotide complementary to a particular mRNA is referred to herein as being “directed against” the product of translation of that message. It is believed that an antisense oligonucleotide, by hybridizing to the RNA and forming a complex, blocks target mRNA ribosomal binding causing translational inhibition. Alternatively, the duplex that is formed by the sense and antisense molecules may be easier to degrade. Other theories describe complexes that antisense RNA could form with complementary DNA to inhibit mRNA transcription. Thus, an antisense oligonucleotide might inhibit the translation of a given gene product by either directly inhibiting translation or through inhibition of transcription.

The invention is discussed in more detail in the Examples below, solely for the purpose of description and not by way of limitation. For clarity of discussion, the specific procedures and methods described herein are exemplified using a murine model; they are merely illustrative for the practice of the invention. Analogous procedures and techniques are equally applicable to all mammalian species, including human subjects.

The following non-limited examples provide methods for significantly improving engraftment of bone marrow in conditioned recipients, while simultaneously reducing the total conditioning required. All scientific and technical terms have the meanings as understood by one with ordinary skill in the art. The methods may be adapted to variation in order to produce compositions or devices embraced by this invention but not specifically disclosed. Further variations of the methods to produce the same compositions in somewhat different fashion will be evident to one skilled in the art.

### EXAMPLES

Materials and Methods

Animals. Male C57BL/10SnJ (B10; H-2^{b}), B10.BR/SgSnJ (B10.BR; H-2^{k}), and BALB/cJ (BALB/c; H-2^{d}) mice were purchased from Jackson Laboratory (Bar Harbor, Me.). Animals were housed in the barrier facility at the Institute for Cellular Therapeutics and cared for according to National Institutes of Health animal care guidelines.

Preparation of allogeneic chimeras. Recipient B10 mice were treated with a single dose of TBI (101.4 cGy/min) from a cesium source (Gamma-cell 40; Nordion, Ontario, Canada). Bone marrow was prepared by a modification of the method previously described (4,11). Briefly, B10.BR donor mice were euthanized and tibias and femurs were harvested. Bone marrow was expelled from the bones with Media 199 (GIBCO-BRL, Grand Island, N.Y.) containing 10 μg/ml Gentamicin (GIBCO-BRL), referred thereafter as chimeric medium (CM). The bone marrow was resuspended in CM by gentle aspiration through an 18-gauge needle. The cells were filtered through sterile nylon mesh with 100 μm pores, centrifuged at 1000 rpm for 10 min at 4° C., and resuspended in CM. A cell count was performed and the cells were diluted to the appropriate concentration to allow injection of 1 ml per animal. Animals were transplanted with 1 ml CM containing 15×10⁶, 30×10⁶ or 45×10⁶ B10.BR bone marrow cells (BMC) via lateral tail vein injection using a 27-gauge needle at 0 hr, 6 hr or on day 1, 2, 3, 4, 5, 8 and 12 post TBI.

Characterization of chimeras. Recipients were characterized for chimerism using flow cytometry to determine the relative percentages of HSC-derived lineages post BMT and then monthly. Peripheral blood was obtained through tail vein bleeding and stained with antibodies specific for MHC class I antigens of donor (PE conjugated anti-H2K^{k}, 36-7-5, mouse IgG₂ₐ) and recipient (FITC conjugated anti-H2K^{b}, AF6-88.5, mouse IgG₂ₐ) origin as previously described. Multi-lineage engraftment was assessed by two-color staining for anti-donor specific antibody (H2K^{k}) and fluorescein-conjugated makers of different lineage including T cells (anti-CD4, RM405; anti-CD8α, 53-6.7; and anti-TCR-βH57-597, B cells (anti-B220, RA3-6B2), NK cells (anti-NK1.1, PK136), and myeloid cells (anti-GR-1, RB6-8C5 and anti-MAC-1, M1/70). All antibodies were purchased from PharMingen (San Diego, Calif.).

Islet isolation. Mouse islets were isolated from the donor pancreas by a modification of the automated method for human pancreatic islet isolation. Briefly, the pancreatic duct of the mouse was cannulated and 2 ml of Hanks solution containing 0.5 mg/ml collagenase (Boehringer-Mannheim, type P, Indianapolis, Ind.) was injected. The distended pancreata were incubated in a 37° C. water bath for 16 min to separate islets. The islets were then purified by centrifugation at 2000 rpm for 21 min on Eurocollins-Ficoll gradients (density=1.108 and 1.069). The purified islets were taken from the gradient interface and resuspended in Hanks Balanced Salt Solution (GIBCO-BRL) containing penicillin and streptomycin for transplantation.

Islet transplantation. Recipients were rendered diabetic through treatment with streptozocin (200 mg/kg intravenously; Upjohn, Kalamazoo, Mich.) at least one week before transplantation. Mice with positive urine glucose test at least 3 consecutive days were used as recipients of islet allografts. Each animal received approximately 600 mouse islets transplanted beneath the left renal capsule.

Criteria for rejection. Recipients of islet allografts were followed by the urine glucose test daily for the first 2 to 4 weeks and then weekly thereafter. Rejection was considered to have occurred when the urine glucose test turned positive. Histological studies were performed on the kidney and native pancreas after rejection of allografts had occurred or after planned nephrectomy and pancreatectomy in urine glucose negative recipients. The tissues were fixed in 10% buffered formalin, stained with hematoxylin and eosin, and processed for light microscopy.

Characterization of host cell population post TBI. To assess the changes in recipient marrow microenvironment at the different time points after TBI, the absolute number of T cells, B cells, NK cells, and NK/T cells in each mouse was determined. Briefly, recipient B10 mice were irradiated with 500 cGy TBI. Bone marrow was harvested on day 0, 1, 2, 3, 4 or 5 post-TBI and a single cell suspension was prepared as described above. The bone marrow lymphocytes were isolated using density centrifugation with Lympholyte-M Separation Medium (Cedarlane Laboratories, Hornby, Ontario, Canada) and washed twice in CM. Marrow from day 3 through 5 was pooled from 2 animals for analysis due to low numbers. Depending on cell recovery, between 0.2-1×10⁶ cells were stained and analyzed by FACS. Cells were first incubated with normal mouse serum to eliminate nonspecific binding. The phenotypic analysis was performed by four-color flow cytometry using anti-B cell (B220), T cell (αβ-TCR, γδ-TCR, CD3ε, CD4 and CD8), and NK cell (NK1.1) mAbs. All mAbs were purchased from PharMingen.

Mixed lymphocyte reaction (MLR). MLR were performed as previously described (14). Briefly, B10 mice were irradiated with 500 cGy TBI. Spleens were harvested at 1 hr, 2 days or 4 days post TBI and a single cell suspension was prepared. Splenocytes were isolated using density centrifugation with Lympholyte-M Separation Medium (Cedarlane Laboratories, Hornby, Ontario, Canada) and washed twice in DMEM medium (Gibco, Grand Island, N.Y.). Splenocytes were resuspended in DMEM supplemented with 5% FBS, 1 mM sodium pyruvate, 2 mM L-glutamine, 10 mM HEPES buffer solution, 0.137M L-Arginine HCL, 1.36 mM/0.027 M Folic Acid/L-Asparagine, 100 U/ml penicillin, 100 μg/ml streptomycin (all from Gibco BRL), 0.05 mM 2-ME (Sigma Chemical Co., St. Louis, Mo.). 1×10⁵ responder cells were cultured 1:1 with irradiated stimulator cells (2000 cGy) in triplicate for 5 days at 37° C. in 5% CO₂. Each well was pulsed with 1 μCi [³H] thymidine (DuPont NEN, Boston, Mass.) 16 hours before harvesting with an automated harvester (PHD Cell Harvester Technology, Cambridge, Mass.) and counted in a β-plate (PerkinElmer Life Science, Gaithersburg, Md.).

Cytokine enumeration. B10 mice were conditioned with 500 cGy TBI. Serum from 3 mice per group was collected at 6 hours, 2 days or 4 days post TBI and stored at −20° C. The evaluation of cytokines and chemokines was performed by Linco Diagnostic Services (St. Charles, Mo.) using the Luminex¹⁰⁰™ multiple immunoassay for mouse cytokines. Data were provided in pg/ml for each sample run in duplicate.

Statistical Analysis. Data are presented as Average±Standard Deviation (SD). A two tailed, non-paired t-test (unequal variances) was used to evaluate statistical differences. The difference between groups was considered to be significant if P<0.05.

Alternatively, a recipient may be conditioned using anti-αβTCR and/or anti-γδTCR monoclonal antibodies for a bone marrow transplant. Hematopoietic stem cell (HSC) chimerism induces tolerance for solid organ allografts. The clinical application of this technique is limited by the morbidity and mortality of fully ablative conditioning. Conditioning of the recipient can be achieved with anti-lymphocyte globulin (ALG) (day−3); 300 cGy TBI (day 0) followed by a single dose of cyclophosphamide (CyP) (day +2) resulted in durable chimerism in MHC plus minor antigen disparate mice. In this embodiment, monoclonal antibodies (mAb) directed against αβ or γδ T-cells are administrated to mice to define which cells in the recipient must be depleted for allogeneic engraftment to result. B10 recipients (H2K^{b}) were pretreated i.v. with 100 mg of anti-αβTCR alone, anti-γδTCR alone and both of mAb on day −3. On day 0, recipients were conditioned with 0, 100, 200 or 300 cGy and transplanted with 15×10⁶ B10.BR (H2K^{k}) bone marrow cells followed by 200 mg/kg i.p. CyP on day +2. Upon conditioning the recipient in this manner the bone marrow may be later infused up to a period of eight days.

In another embodiment conditioning of the recipient may be achieved using anti-αβTCR and anti-CD8 monoclonalantibodies. In this embodiment, recipient B57BL/10 (H2^{b}) mice were pretreated in vivo with mAbs anti-αβ-TCR and anti-CD8 3 days prior to TBI with 0, 100, 200 or 300 cGy TBI TBI followed by the transplantation of 15×10⁶ allogeneic (B10.BR; H2^{k}) marrow cells, up to eight days after the recipient is conditioned.

A wide variety of uses are encompassed by the invention described herein, including, but not limited to, the conditioning of recipients by non-lethal methods for bone marrow transplantation in the treatment of diseases such as hematologic malignancies, infectious diseases such as AIDS, autoimmunity, enzyme deficiency states, anemias, thalassemias, sickle cell disease, and solid organ and cellular transplantation.

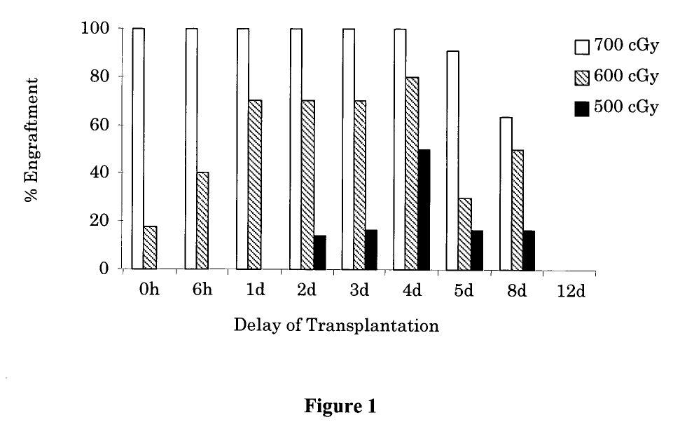

A delay in marrow infusion improves engraftment. As shown if FIG. 1, a dose titration of TBI was preformed to evaluate the influence of timing of BMT on engraftment. When B10 recipients were conditioned with 700 cGy and reconstituted with 15×10⁶ B10.BR BMC, 100% of recipients engrafted when the marrow was infused between day 0 and day 4 relative to the TBI, and 90% and 60% engrafted when the marrow was infused on day 5 and 8, respectively. When 600 cGy TBI was administered, 70-80% of recipients engrafted at 1 month post BMT when the marrow was infused between 1 to 4 days following the conditioning. In contrast, 40% of recipients engrafted when the marrow was infused with a 6 hr delay, and only 17% when the marrow was infused immediately after the TBI conditioning. With 500 cGy TBI, engraftment occurred only when the BMT was carried out 2 to 8 days after conditioning. The highest proportion (50%) of engraftment occurred at 4 day's delay. These data show that a delay of donor marrow infusion following TBI administration significantly improves allogeneic engraftment in nonmyeloablated recipients. The data in Table 1 below, indicates the levels of donor chimerism one month after BMT.

TABLE 1

| TBI | DELAY OF BM INFUSION | | | | | | | | |
|---|---|---|---|---|---|---|---|---|---|
| (cGy) | 0 H | 6 H | 1 D | 2 D | 3 D | 4 D | 5 D | 8 D | 12 D |
| 700 | N = 6 | N = 6 | N = 6 | N = 7 | N = 6 | N = 6 | N = 11 | N = 11 | N = 10 |
| | 6 × 100% | 6 × 100% | 90.0%, | 99.0% | 6 × 100% | 6 × 100% | 0 | 4 × 0 | 10 × 0 |
| | | | 5 × 100% | 6 × 100% | | | 97.2%, | 41.9% | |
| | | | | | | | 9 × 100% | 6 × 100% | |
| 600 | N = 6 | N = 15 | N = 10 | N = 10 | N = 10 | N = 10 | N = 10 | N = 10 | N = 10 |
| | 5 × 0 | 9 × 0 | 3 × 0, | 3 × 0 | 3 × 0 | 2 × 0 | 7 × 0 | 5 × 0 | 10 × 0 |
| | 98.6% | 6.1% | 94.1% | 46.9% | 1.8% | 90.6% | 16.0% | 50.5% | |
| | | 23.3% | 6 × 100% | 6 × 100% | 6 × 100% | 7 × 100% | 2 × 100% | 84.2% | |
| | | 4 × 100% | | | | | | 3 × 100% | |
| 500 | N = 6 | N = 6 | N = 6 | N = 7 | N = 6 | N = 6 | N = 6 | N = 6 | N = 6 |
| | 6 × 0 | 6 × 0 | 6 × 0 | 6 × 0 | 5 × 0 | 3 × 0 | 5 × 0 | 5 × 0 | 6 × 0 |
| | | | | 100% | 2.0% | 4.6% | 18.7% | 52.9% | |
| | | | | | | 68.5% | | | |
| | | | | | | 95.8% | | | |

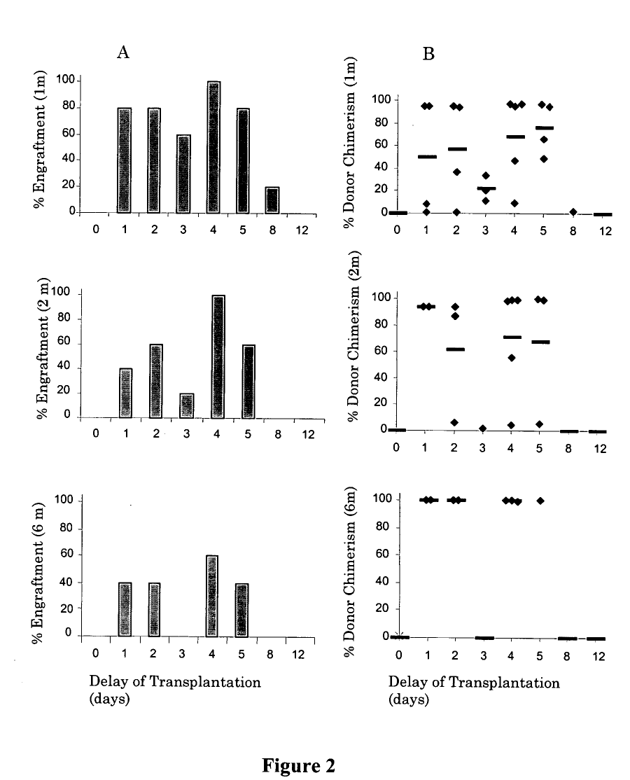

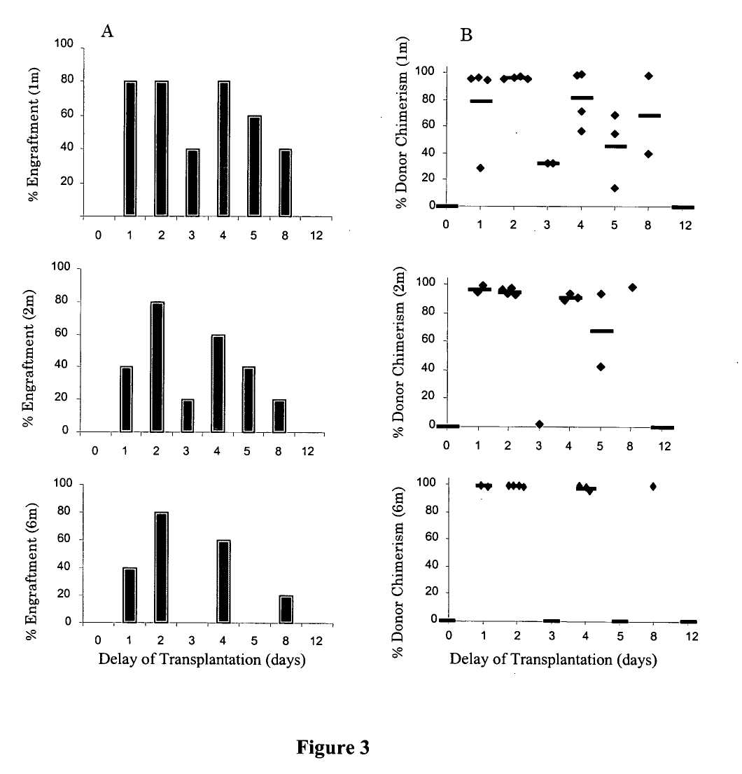

Influence of cell dose on engraftment. Next, the influence of cell dose on engraftment was investigated when the marrow was infused at certain time points following nonmyeloablative conditioning. Recipients were conditioned with 500 cGy TBI and transplanted with 30×10⁶ or 45×10⁶ donor BMC. No engraftment occurred when the BMC were given on day 0 or after day 8 when the donor BMC doses were increased to 30×10⁶ and 45×10⁶. The highest percentage of animals with engraftment occurred with a 4 day delay in animals receiving 30×10⁶ BMC, see FIG. 2A, and a 2 or 4 day delay in animals receiving 45×10⁶ BMC as shown in FIG. 3A. Engraftment improved significantly with an increase of BMC dose from 15×10⁶ to 30×10⁶ and 45×10⁶ BMC 1 month post transplantation when the marrow infused on day +4, see FIGS. 2 and 3. The level of chimerism ranged from 1.2% to 99% donor cells 1 month post BMT (FIGS. 2B and 3B). The percentage of chimeras with durable engraftment at 6 months was 60% in recipients of either 45×10⁶ or 30×10⁶ bone marrow cells infused on day 4 (FIGS. 2A and 3A). Similarly, 80% of recipients of 45×10⁶ versus 40% of recipients of 30×10⁶ bone marrow cells were still chimeric at 6 months when the marrow was infused on day +2. In the majority of the animals that exhibited durable engraftment, the percentage donor chimerism increased to reach nearly 100% by 6 months. One animal died 3 months post BMT with 93.7% donor chimerism. There was no evidence for GVHD in any of the animals. It is of note that the percentage of animals engrafting was much greater on day 2 and 4 compared with day 3 for all treatment groups. Moreover, if the marrow was infused on day 3, the durability of engraftment was significantly impaired compared to day 2 and day 4, with 0 recipients chimeric at 6 months.

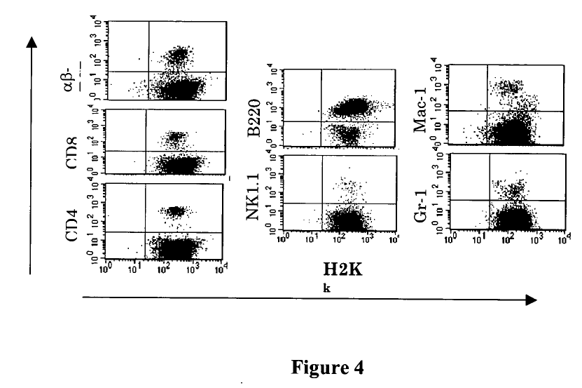

Evidence for multilineage chimerism. To determine whether the chimerism was the result of engraftment of the pluripotent HSC, peripheral blood from chimeras was analyzed for the presence of various stem cell-derived hemolymphopoietic lineages of donor origin using two-color flow cytometry. In all of the 6 animals with durable donor chimerism, lymphoid lineages (T and B cells), NK cells, and myeloid lineages (macrophages and granulocytes) produced by stem cells of donor origin were detected. See, FIG. 4.

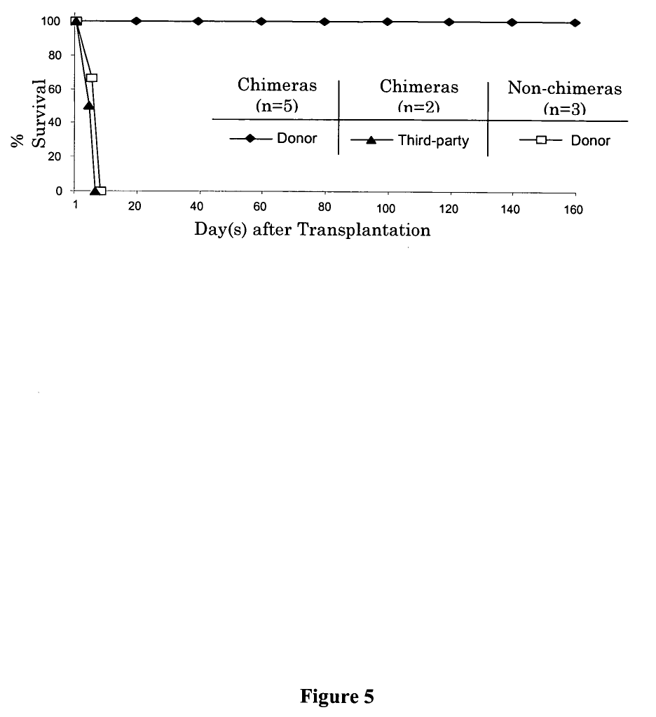

Survival of islet allografts in chimeras. The allogeneic chimeras used in islet transplantation were prepared using 500 cGy TBI and transplanted with 30 or 45×10⁶ bone marrow cells on day +1 to +8. Diabetes was induced in chimeras and in controls that had initially engrafted but subsequently lost their chimerism using the islet-toxic drug streptozocin 4 to 6 months post BMT. Mice that exhibited sustained glucosuria for at least three consecutive days were transplanted with donor-specific or MHC-disparate third party islets. Survival of donor-specific B10.BR islet allografts was significantly prolonged in chimeras (median survival time-MST>160 days, shown in FIG. 5). One chimera died at 133 days post islet transplantation with a negative urine glucose test. MHC-disparate third-party BALB/c grafts were rapidly rejected by the chimeras (MST=6 days, n=2). The grafts were retrieved at 160 days. All donor-specific islet allografts examined histologically (n=4) showed viable islets with normal morphology, confirming that transplanted islets were functional and the source of insulin production. In contrast, the control animals that exhibited chimerism at month 1, but had become non-chimeric by the time of islet transplantation, promptly rejected donor-specific B10.BR islets (MST=7.5 days, n=3).

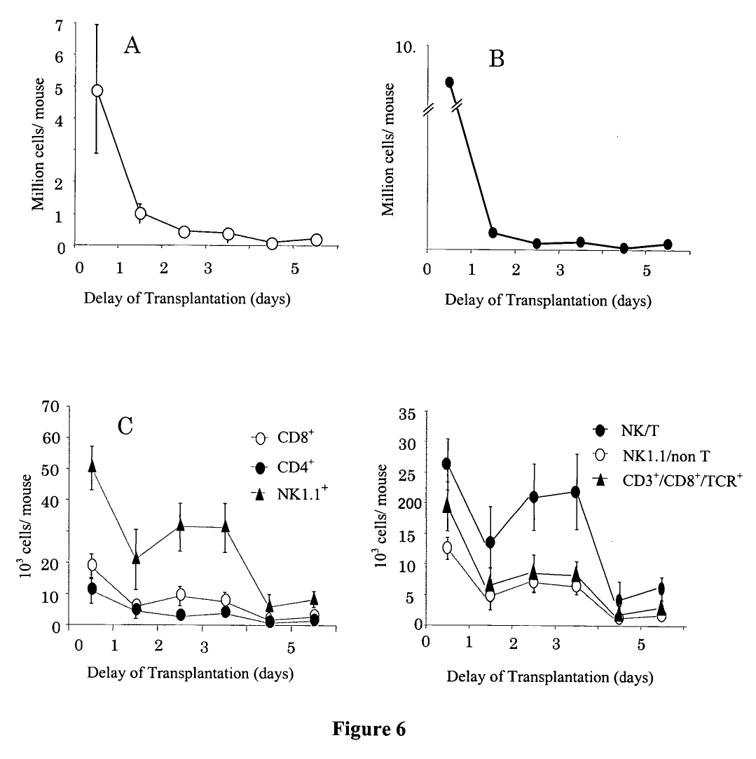

Correlation of recipient microenvironment with outcome. In order to define the relative contribution of specific cell subsets in the host microenvironment to graft rejection, we conditioned recipients with 500 cGy TBI and characterized the cellular subpopulations remaining on day 0, 1, 2, 3, 4 and 5 post conditioning. The total absolute number of total bone marrow cells prepared from femurs and tibia declined steadily following 500 cGy TBI (FIG. 6A), reaching its lowest point on day 4. The most dramatic reduction occurred for B cells (B220⁺) as early as day 1 (FIG. 6B), decreasing from 8 million to less than 100,000 on day 1 and reaching a nadir of 30,000 on day 4 after irradiation. In contrast, the decrease in CD4, CD8, and NK1.1 positive cells occurred gradually, with the lowest absolute numbers at day 4 (FIG. 6C). The greatest reduction of conventional T cells occurred by day 1 (FIG. 6C). Interestingly, the number of NK cells initially declined, increased on day 2-3, then significantly dropped on day 4 (P<0.05) (FIG. 6C).

The subfamilies of NK cells that have been determined to play a role in alloresistance to engraftment were also interrogated. NK1.1⁺/CD3ε⁻ and CD3ε⁺/CD8⁺/NK1.1⁺ cells decreased within 24 hr following the TBI, then rebounded slightly on day 2 and 3, and finally reached a nadir on day 4 (FIG. 6D). The αβ-TCR⁺/NK1.1⁺ population declined more slowly, with a slight increase on day 2 and 3, then a nadir to nearly 0 on day 4. The proportion of these cell populations on the various days directly correlated with the percentage of animals with donor engraftment.

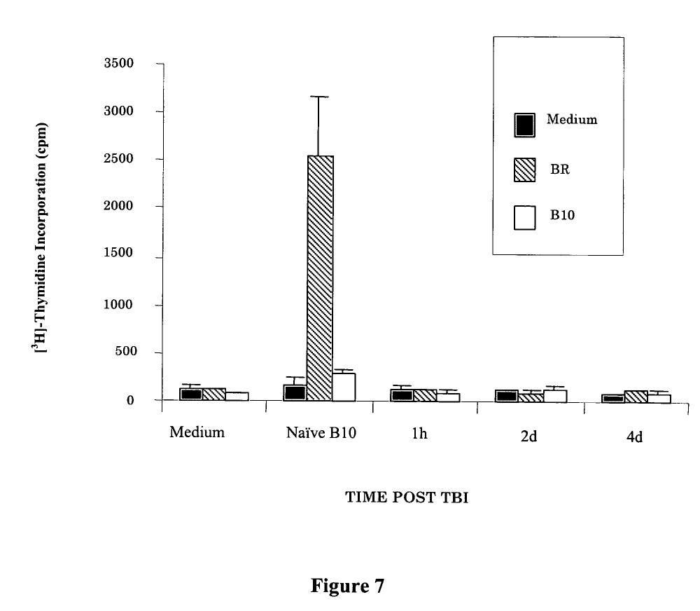

Mixed lymphocyte reactivity is significantly impaired following TBI. To evaluate the influence of conditioning on anti-donor reactivity, B10 mice were conditioned with 500 cGy TBI and MLR reactivity of splenic lymphoid cells assessed at 1 hour, 2 days, and 4 days thereafter. All proliferative activity was significantly reduced following conditioning as shown in FIG. 7.

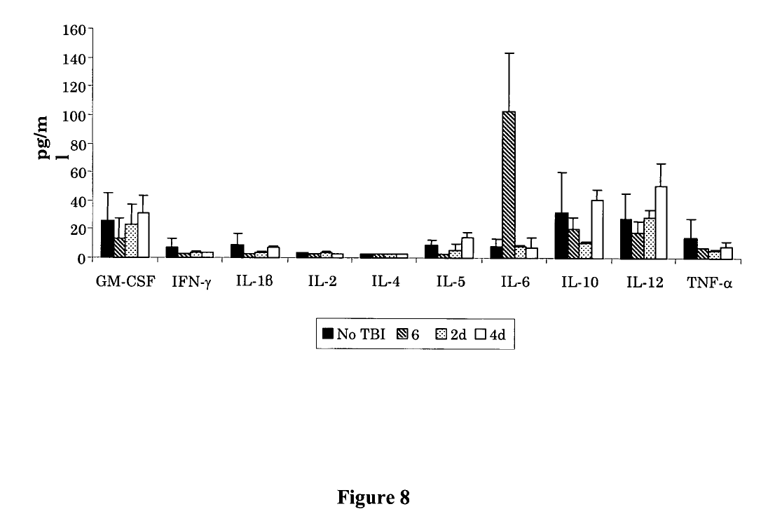

IL-6 production is significantly increased early following TBI and the level inversely correlates with outcome. The recipient cytokine milieu was evaluated at 6 hours, 2 days, and 4 days following conditioning with 500 cGy TBI shown in FIG. 8. Most notably, IL-6 production was significantly increased early following conditioning (FIG. 8), while other pro-inflammatory cytokines including IL-2, IFN-γ, TNF-α, and IL-12 did not correlate with the enhanced engraftment observed at day +4. It is of note that GM-CSF, IL-5, IL-1B, and IL-12 levels were greater on day 4 than day 2 or 6 hours following the TBI.

Although the association between hematopoietic chimerism and tolerance has been recognized for over 50 years the 7-10% regimen-related mortality associated with conventional bone marrow transplantation has dampened enthusiasm for applying this approach clinically to induce tolerance to allografts. Nonmyeloablative conditioning strategies to achieve engraftment have begun to extend the therapeutic application of BMT far beyond life-threatening illnesses by reversing the risk:benefit ratio associated with BMT. Engraftment of allogeneic marrow has now been achieved in recipients conditioned with low-dose irradiation in conjunction with cytoreductive agents and nonspecific immunosuppressive agents (6,10,20). Chimeras prepared in this fashion exhibit donor specific tolerance in vitro and in vivo similar to fully ablated recipients. As little as 1% donor chimerism is sufficient to induce donor-specific transplantation tolerance. With this recognition, clinically applicable strategies to intentionally establish mixed chimerism with less aggressive conditioning have been explored.

Two conditions are required for engraftment of stem cells in allogeneic BMT: 1) cytoreduction to prepare vacant niches within the host marrow, and 2) immunosuppression to prevent the rejection of the donor marrow. Most clinical BMT conditioning regimens use TBI in combination with immunosuppressive agents. Low dose TBI is required to achieve marrow engraftment even in syngeneic recipients. Studies by Down, et al. demonstrated that for syngeneic engraftment, the minimum radiation dose required to induce durable mixed donor/host chimerism was≧200 cGy TBI. The group hypothesized that specific cellular populations in the hematopoietic microenvironment with a radiation sensitivity within this range were influencing the niches available for the transplanted marrow to home. Significantly more (700 cGy) of TBI is required for engraftment when the donor is MHC disparate to the recipient. However, the TBI dose can be decreased to as low as 200 cGy TBI if immunosuppressive agents are administered in combination with the TBI. A precise definition of the role of conditioning in enhancing engraftment will allow a more specific approach to establishing chimerism to induce tolerance.

It has been conventionally believed that repair after irradiation and conditioning begins almost immediately. If this were the case, the time constraints imposed upon clinical protocols utilizing cadaver marrow would be more limited, since the isolation of a single cell suspension from vertebral marrow takes approximately up to 24 hours. In addition, the influence of timing with respect to marrow infusion following nonmyeloablative conditioning could differ from ablated recipients. Sykes, et al., previously demonstrated that engraftment could be achieved when the administration of a mixture of T cell depleted (TCD) syngeneic plus allogeneic marrow is delayed by 8 days following ablative TBI. Similarly, Xun, et al., reported that a 4 day delay between ablative conditioning and infusion of marrow plus splenocytes was associated with a decreased mortality from GVHD. However, a systematic evaluation of the influence of a delay in marrow infusion on outcome had not been performed. The present invention demonstrates for the first time that a delay in marrow infusion is associated with a decrease in the amount of conditioning required and improved engraftment. Surprisingly, it has been discovered that for a given dose of TBI, a delay in transplantation of the marrow for up to 8 days, and preferably 5 days, following conditioning was associated with a higher proportion of animals engrafting. For a given dose of conditioning, the highest proportion of animals engrafted when the marrow infusion was delayed by 4 days. In recipients conditioned with 700 cGy of TBI, a delay of marrow infusion for up to 4 days did not impair the establishment of chimerism. In recipients treated with 600 cGy TBI, engraftment was significantly improved if the marrow was infused 1-4 days following conditioning compared to the conventional delay of approximately 6 hours. Strikingly, only 17% of recipients engrafted if the marrow was infused immediately after conditioning. Similarly, in recipients conditioned with 500 cGy TBI, engraftment did not occur at day 0 or day 1, but only if the marrow was infused 2-8 days following conditioning. Taken together, these data suggest that some radiation-sensitive host effector cells remain functional for a period of time following exposure to irradiation. This explanation is less likely in light of the lack of proliferative capability in MLR at all time points. The spontaneous release of inflammatory cytokines immediately following the conditioning that would impair engraftment may be an alternative explanation. This mechanism is supported by a significant increase in IL-6 immediately following the administration of TBI, followed by a subsequent decline over time. IL-6 is a proinflammatory cytokine that has been shown to correlate with transplant-related morbidity after BMT. However, a role for this cytokine in impairing engraftment has not been previously noted. It is of note that other pro-inflammatory cytokines, including IL-2, IFN-γ, TNF-α, and IL-12 did not correlate with the kinetic for engraftment. Notably, GM-CSF, IL-5, IL-1B, and IL-12 levels were greater on day 4 compared to day 2 and 6 hours following TBI.

A dramatic drop in absolute bone marrow cell numbers was observed within 1 day after conditioning with 500 TBI. The cell numbers consistently fell to nadir on day 4 after radiation and started to increase on day 5. Coincident with this observation, engraftment inversely correlated with cellularity of the microenvironment, being highest on day 4. Interesting, for all treatment groups there was a significant decrease in the proportion of animals engrafting as well as durability of engraftment if the marrow was infused on day 3. In light of the initial decrease and the subsequent rise in NK/T cells day 2-3, we hypothesize that residual host stem cells may rapidly produce some additional subsets of effectors cells (i.e., NK/T) until tolerance is induced, given the fact that NK/T cells appear within 24 hr following allogeneic BMT in ablated recipients. It is of note that on day 4, the optimal percentage of engraftment that occurred following a given dose of partial conditioning corresponded with a nadir in the absolute number of NK/T and NK cells. Additionally, on day 3, impaired engraftment also corresponded with the peak for production of NK/T cells. Studies are in progress to evaluate the role of host NK cells in engraftment.

No engraftment was observed when the BMT was performed>8 days following conditioning, probably due to the re-emergence of endogenous hematopoiesis with the recovery of cell populations which occupy niches and/or actively resist donor engraftment. However, this repair occurs much less rapidly than anticipated.

The present invention suggests that delayed infusion of marrow in partially conditioned recipients results in improved engraftment for a given dose of TBI. As the mechanism by which conditioning, engraftment, and induction of tolerance occurs, one can theoretically identify the optimal timing and strategy for conditioning. Moreover, by specifically targeting those residual effector cells and/or cytokines for conditioning, one could optimize a minimal approach for partial conditioning. As a result, the widespread clinical application of BMT to induce tolerance would be one step closer to reality.

The foregoing description is considered as illustrative only of the principles of the invention. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. Furthermore, since a number of modifications and changes will readily occur to those skilled in the art, it is not desired to limit the invention to the exact construction and process shown described above. Accordingly, all suitable modifications and equivalents may be resorted to falling within the scope of the invention as defined by the claims which follow.

1. A method for conditioning a recipient for bone marrow transplantation comprising subjecting said recipient to a composition that specifically depletes αβ-, and γδ-TCR⁺ T cells and/or CD8⁺ T cells in the recipient hematopoietic microenvironment, followed by a delayed transplantation with a donor cell preparation containing hematopoietic stem cells from a donor that are matched at the major histocompatibility complex class I K locus with the recipient hematopoietic microenvironment.

2. The method of claim 1 in which said composition comprises antibodies specific for αβ-, and γδ-TCR⁺ T cells and/or CD8⁺ T cells.

3. The method of claim 1 in which said composition comprises antisense DNA that is directed against the precursors of αβ-, and γδ-TCR⁺ T cells and/or CD8⁺ T cells.

4. The method of claim 3 wherein antisense DNA alters the translation of the α-chain, β-chain, γ-chain, or δ-chain of TCR⁺ T cells.

5. The method of claim 3 wherein antisense DNA alters the transcription of the α-chain, β-chain, γ-chain, or δ-chain of TCR⁺ T cells.

6. The method of claim 1 in which said composition a cytotoxic drug specific for αβ-, and γδ-TCR⁺ T cells and/or CD8⁺ T cells.

7. The method of claim 1 wherein the recipient is further conditioned by subjecting the recipient to a total dose of total body irradiation of less than or equal to 300 cGy.

8. The method of claim 1 wherein the recipient is further conditioned by subjecting the recipient to an alkylating agent.

9. The method of claim 8 wherein said alkylating agent is cyclophosphamide.

10. The method of claim 1 wherein said composition specific to αβ-, and γδ-TCR⁺ T cells and/or CD8⁺ T cells in the recipient hematopoietic microenvironment totally eliminates said cells from the recipient hematopoietic microenvironment.

11. The method of claim 1 wherein the delay of transfusion is a period of time between 0 and 8 days.

12. The method of claim 11, wherein said period of time is up to five days.

13. A method for transplanting bone marrow comprising,
(1.) conditioning a recipient for bone marrow transplantation comprising subjecting said recipient treatment with a total dose of total body irradiation from 100 to 300 cGy, and treating the patient with a composition that specifically depletes αβ-, and γδ-TCR⁺ T cells and/or CD8⁺ T cells in the recipient hematopoietic microenvironment;
(2.) delaying transplantation with a donor cell preparation by up to 8 days.

14. The method of claim 13 wherein the recipient is further treated with an alkylating agent before, during, or after exposure to said composition that specifically depletes αβ-, and γδ-TCR⁺ T cells and/or CD8⁺ T cells in the recipient hematopoietic microenvironment.

15. The method of claim 14 wherein said alkylating agent is cyclophosphamide.

16. A method of partially or completely reconstituting a mammal's lymphohematopoietic system comprising administering to the mammal a composition that specifically depletes αβ-, and γδ-TCR⁺ T cells and/or CD8⁺ T cells in the recipient hematopoietic microenvironment, followed by a 0 to 8 delay of transplantation with a donor cell preparation containing hematopoietic stem cells from a donor that are matched at the major histocompatibility complex class I K locus with the recipient hematopoietic microenvironment.

17. The method of claim 16, in which the mammal suffers from autoimmunity.

18. The method of claim 17 in which the autoimmunity is diabetes.

19. The method of claim 17, in which the autoimmunity is multiple sclerosis.

20. The method of claim 17, in which the autoimmunity is sickle cell.

21. The method of claim 17, in which the autoimmunity is anemia.

22. The method of claim 17, in which the mammal suffers from a hematologic malignancy.

23. The method of claim 16, in which the mammal requires a solid organ or cellular transplant.

24. The method of claim 16, in which the mammal suffers from immunodeficiency.

25. The method of claim 16, wherein said delay is up to 5 days.

25. A method for conditioning a recipient for bone marrow transplantation comprising subjecting said recipient treatment with a total dose of total body irradiation from 100 to 300 cGy, and treating the patient with a composition that specifically depletes αβ-TCR⁺ T cells and/or CD8⁺ T cells in the recipient hematopoietic microenvironment, transplanting with a donor cell preparation containing hematopoietic stem cells from a donor that are matched at the major histocompatibility complex class I K locus with the recipient hematopoietic microenvironment.

26. The method of claim 25, wherein said transplanting occurs between 0 and 8 days following total body irradiation.

27. The method of claim 25, wherein said transplanting occurs between 2 and 4 days following total body irradiation.

28. A method for conditioning a recipient for bone marrow transplantation comprising subjecting said recipient to a total dose of total body irradiation from 100 to 700 cGy, and infusing the recipient with a donor cell preparation containing hematopoietic stem cells from the donor between 0 and 8 days following total body irradiation.

29. The method of claim 28, wherein said dose of total body dose irradiation is from 100 to 500 cGy and infusion occurs between 1 and 5 days following total body irradiation.

30. The method of claim 28, wherein said dose of total body dose irradiation is from 100 to 300 cGy and infusion occurs between 1 and 3 days following total body irradiation.
